(19) United States
(12) Patent Application Publication
(10) Pub. No.: US 2008/0114327 A1
(43) Pub. Date: May 15, 2008

(54) MOISTURE CONTROL UNDERGARMENT

(76) Inventor: Carol Barge, San Clemente, CA (US)

Correspondence Address: Vista IP Law Group LLP, 2040 MAIN STREET, 9TH FLOOR, IRVINE, CA, 92614, US

(21) Appl. No.: 11/558,442

(22) Filed: Nov. 9, 2006

(51) Int. Cl. A41B 9/12; A61F 13/15

(52) U.S. Cl. 604/396; 2/400

(57) ABSTRACT

A reusable, launderable, moisture control undergarment comprises a brief portion made mostly of a first material. The brief portion generally includes a waist opening, two leg openings and a crotch area. A moisture protection pad is disposed in at least the crotch area of the brief portion. The moisture protection pad comprises a first layer formed of a non-woven, absorbent material, and a second layer which is moisture impermeable. The second layer is disposed on the outer side of the first layer and covers substantially the entire outer side of the first layer. The second layer may be comprised of the first material which has been made moisture impermeable through a Teflon® treatment. A third layer formed of the first material is disposed over inner side of the first layer.

### FIELD OF THE INVENTION

The present invention relates generally to protective undergarments, and more particularly to a reusable protective undergarment having a discreet moisture control pad for containing moisture, such as that caused by perspiration, incontinence or other urinary or genital conditions.

### BACKGROUND OF THE INVENTION

The present invention relates generally to the control of moisture from bodily fluids in the pelvic area of the adult human body. Such moisture has several different sources and causes ranging from normal bodily functions to medical problems caused by medical conditions and illness. For instance, moisture in the pelvic area may be caused by conditions as normal and common as perspiration or female menstrual flow, or less common conditions such as incontinence and loss of bladder or bowel control caused by aging or other medical conditions. Excessive, uncontrolled moisture in the pelvic area can be the source of not only discomfort and irritation, but also embarrassment, bothersome clean-up and stained/damaged clothing.

In order to manage moisture in the pelvic area, various devices and undergarments for have been previously marketed and/or proposed. As some of the most common examples, there are female hygiene products such as tampons and pads, and there are adult diapers, sometimes called protective underwear. In general, these products are disposable and are designed to address moderate to heavy moisture problems. As a result, these products tend to be rather thick and bulky, and even thin feminine pads are visible under close fitting clothing.

A number of reusable, launderable, moisture management products have also been marketed and/or proposed. As an example, U.S. Pat. No. 4,044,769 discloses a women's panty having a pocket for holding a sanitary pad and an opening on the inside wall of the compartment for allowing fluid form the wearer to contact the pad. However, this panty still requires a disposable insert which must be purchased making this solution more expensive and environmentally unfriendly.

U.S. Pat. No. 6,610,901 appears to disclose a moisture control undergarment which is fully reusable and launderable. The undergarment includes a crotch area comprising a lamination consisting of two laminae. The first, inner lamina is a liquid permeable fabric and the second lamina is a liquid impermeable but vapor permeable material. The vapor permeable lamina allow moisture to evaporate through the lamination and therefore through the undergarment. In view of this evaporative scheme, the inner lamina is not designed to hold moisture, but is only intended to transmit moisture from the wearer to the second lamina. Thus, this design allows odors and moisture in the form of vapor to escape the undergarment. These odors and vapor can then condense on the wearer's outer clothing causing wet spots and detectable and undesirable smell.

A similar moisture management undergarment is disclosed in U.S. Pat. No. 5,291,617. This patent discloses an undergarment having a multi-layer panel in the crotch area of the undergarment which comprises first and second fabric layers. Each of the first and second fabric layers is constructed of a two layer fabric consisting of one layer of hydrophobic yarns and one layer of hydrophilic yarns. An intermediate wicking insert is provided between the first and second fabric layers for wicking moisture from the crotch area to the waist area of the undergarment. Finally, a liquid impervious, vapor permeable crotch shield is sewn into the inside of the outer shell of the undergarment. This design suffers from the same drawbacks as the undergarment above, in that it does not absorb and contain the bodily moisture but instead wicks the moisture to areas from which it can evaporate. Indeed, the woven fabrics utilized could not contain a significant amount of moisture. In addition, the disclosed design does not provide a liquid impervious layer over the entire pad, such that areas of the first and second fabric layers and the wicking insert are in direct contact with the shell of the undergarment which can allow moisture to leak through to a wearer's outer clothing. Moreover, the numerous layers (at least 6 layers of woven fabric) result in a complicated and relatively thick, bulky pad.

Accordingly, there is a need for an environmentally friendly, reusable, launderable, moisture control undergarment which is unobtrusive and discreet, yet can absorb and contain light amounts of bodily fluids in the crotch area and which prevents penetration of moisture to the exterior of the undergarment and consequently to a wearer's outer clothing.

### SUMMARY OF THE INVENTION

The present invention is directed to a reusable, launderable, moisture control undergarment for managing slight moisture control issues in the pelvic area of a wearer. For instance, the undergarment is designed to control issues such as minor urinary leakage and incontinence, light menstrual flow such as spotting, and vaginal discharge due to sexual activity, infection, or hormonal fluctuations. The undergarment includes an innovative moisture control pad which absorbs and contains bodily fluids in the pelvic area of a wearer and which prevents such bodily fluids from wetting through to the exterior of the undergarment. The pad is designed to be very thin and inconspicuous, yet provides effective containment of the fluids and their odors.

The undergarment comprises a brief portion made mostly of a first material. The brief portion generally includes a waist opening, two leg openings and a crotch area. The first material may form most of the brief portion, including the seat, sides, front and waist. The brief portion may also comprise other materials, such as an elastic waist band, and/or an elastic border around the leg openings. The brief portion can be in the form of any type of undergarment, for example, a women's panty, including styles such as brief, bikini, boyshort, hipster, tanga, thong, etc., pantyhose, or the various types of men's underwear styles such as briefs, boxers, etc. For clarity in describing the present invention, the brief is described to have an inside surface which is in contact with the wearer, and an outside surface which faces outward from the wearer.

A moisture protection pad is disposed in at least the crotch area of the brief portion and may extend continuously from the front side of the crotch area to the underside of the crotch area between the legs, to the seat side (or back side) of the crotch area. The moisture protection pad comprises a first layer formed of a non-woven, absorbent material, and a second layer which is moisture impermeable.

The first layer absorbs the moisture from the wearer's bodily fluids and contains the moisture. The first layer may be formed of a non-woven, absorbent material which is reusable and launderable.

The second layer is disposed on the outer side (the side facing away from the wearer) of the first layer and covers substantially the entire outer side of the first layer. The second layer is formed of a material which is moisture impermeable and preferably vapor impermeable also. The second layer may be formed of a portion of the first material in the crotch area which has been has been made moisture impermeable through a Teflon® treatment (described in more detail below). Alternatively, the second layer may be formed of a second material (different from the first material) which is moisture and vapor impermeable. For instance, the second material may be comprised of a fabric which has been made moisture impermeable through a Teflon® treatment.

In yet another embodiment, the second layer may comprise a separate moisture impermeable layer such as a layer of polymer film. The polymer film may be affixed to the first layer or it may be affixed to the first material of the brief portion in the crotch area. In another embodiment, the polymer film may be heat fused to the first layer or the material of the brief portion. On the other hand, the second layer may be made of separate fabric layer, for instance, formed of the first material, and a protective layer formed of a moisture impermeable material provided on the inner side of the fabric layer. Similar to the above-described embodiment, the protective layer may be a moisture impermeable polymer film which is affixed, and/or heat fused, to the inner side of the fabric layer.

In order to give the pad a look and feel consistent with the rest of the undergarment, the crotch area of the undergarment further comprises a third layer formed of the first material disposed over any exposed portion(s) of the pad. The third layer covers the inner side of the first layer or the outer side of the second layer, depending on the configuration of the pad relative to the brief portion, as discussed in detail below. In this way, the only surfaces in contact with the wearer, including the inside and outside of the undergarment, are comprised of the first material making up most of the brief portion.

In order to make the moisture protection pad relatively inconspicuous and discreet, the pad is configured to have a total thickness of less than 0.15 inch, or alternatively less than 0.13 inch, or alternatively less than 0.11 inch.

One or more of the layers of the moisture protection pad may be permanently affixed to the brief portion by stitching, adhesive, or other suitable means. In the case in which the second layer is at least partially comprised of the material of the brief portion, this simply entails affixing the first layer to the brief portion. If the second layer is not comprised of the material of the brief portion, then both the first and second layers are affixed to the brief portion.

In another aspect of the present invention, the first material is a super micro polyester and spandex blend material. This material provides some degree of elastic stretch, and also has a soft feel.

In still another aspect of the present invention, the first layer is formed of a non-woven, absorbent, blended polyester and viscose material. Moreover, the first layer has an absorption capacity of at least 0.9 ounces of bodily fluid for a single layer of material, or at least 1.4 ounces of bodily fluid for two layers of material, and the absorbency of the first layer does not deteriorate after a reasonable number of washings (for instance, 25 washings).

In yet another aspect of the present invention, the first layer and second layer are formed of materials which have good antimicrobial and odor-reducing properties, further allowing the wearer to feel confident without being self-conscious that the protective undergarment is noticeable by others.

Accordingly, a reusable, launderable, moisture control undergarment which manages light moisture control issues, protects a wearer's outer clothing from leakage, and is comfortable and relatively inconspicuous and discreet.

### BRIEF DESCRIPTION OF THE DRAWINGS

FIG. 1 is front, perspective view of a moisture control undergarment according to the present invention, with the layers partially disassembled.

FIG. 2 is a rear, perspective view of the moisture control undergarment of FIG. 1.

FIG. 3 shows test results for an antibacterial test on a super micro polyester and Spandex blend fabric.

FIG. 4 shows test results for a hot odor test on a super micro polyester and Spandex blend fabric.

FIG. 5 shows test results for a moisture vapor transmission test on a super micro polyester and Spandex blend fabric.

FIG. 6 shows test results for a wicking test on a super micro polyester and Spandex blend fabric.

FIG. 7 shows test results for a water absorption test on a super micro polyester and Spandex blend fabric.

FIG. 8 shows test results for a flammability test on a super micro polyester and Spandex blend fabric.

FIG. 9 shows test results for a dimensional stability on a super micro polyester and Spandex blend fabric.

FIG. 10 shows results for a thickness measurement of a super micro polyester and Spandex blend fabric and the fabric with a heat fused film coating.

FIGS. 11A-11B show test results for various tests on several non-woven, absorbent materials.

FIGS. 12A-12B show the test data for the test results shown in FIGS. 11A-11B.

FIG. 13 shows the test results for an antibacterial test and a thickness measurement on a non-woven, polyester and viscose material.

FIGS. 14A-14B show test results for various tests on a super micro polyester and Spandex blend fabric which has been treated with the Repel Teflon treatment.

FIGS. 15A-15C show test results for various tests on a super micro polyester fabric with a heat fused film coating.

FIGS. 16A-16B show test results for various tests on a complete undergarment according to one embodiment of the present invention.

### DETAILED DESCRIPTION OF THE INVENTION

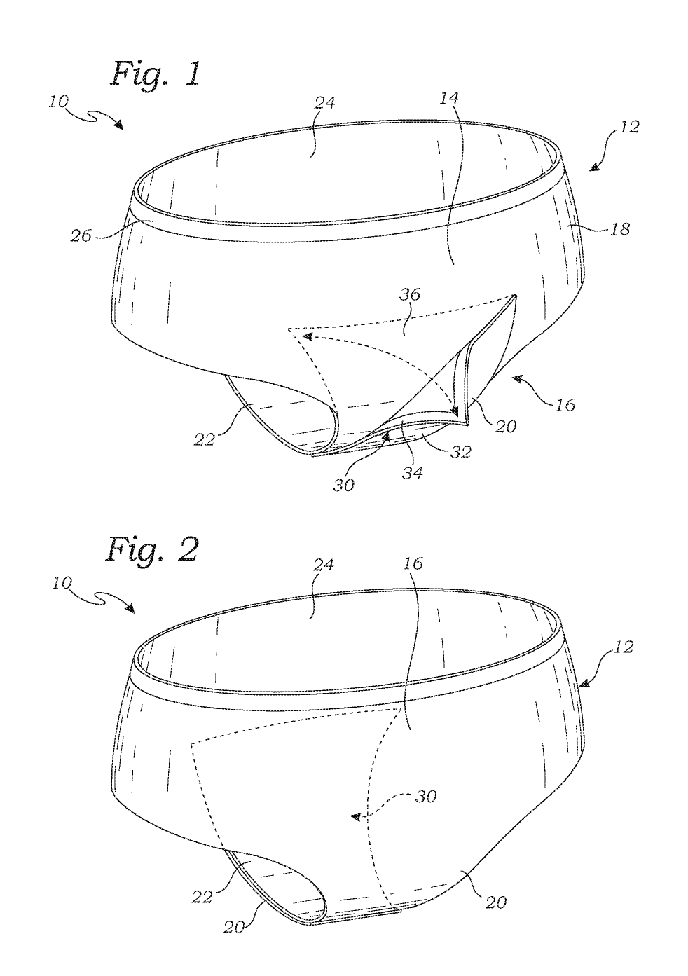

Referring now to FIGS. 1 and 2, an embodiment of a moisture control undergarment 10 according to the present invention is shown. The undergarment 10 comprises a brief portion 12 in the form of a typical panty brief. The brief portion 12 is shown as a typical brief style, but could be any style of panty, underwear, pantyhose, etc. The brief portion 12 includes a front 14, a rear 16, a waist 18, a crotch area 20, two leg openings 22 and a waist opening 24. The front 14, rear 16 and waist 18 may are typically made of the same first material, usually a woven fabric. The first material should be a comfortable material, soft to the touch, and have some elastic stretch to it so that it forms to the wearer's body size and shape. As discussed in greater detail below, the first material may also have certain functional characteristics, including antimicrobial, odor-reducing, permeability and wicking properties.

For purposes of describing the present invention, the undergarment 10 is described to have an inside surface which is in contact with the wearer, and an outside surface which faces outward from the wearer. In addition, reference to a first element being “on the inner side of” a second element means that the first element is positioned toward the inside of the undergarment relative to the second element, and the first and second elements may be directly adjacent to each other or there may be one or more intervening elements between them. Similarly, reference to a first element being “on the outer side of” a second element means that the first element is positioned toward the outside of the undergarment relative to the undergarment second element, and the first and second elements may be directly adjacent to each other or there may be one or more intervening elements between them. In the same vein, the “inner side” of an element is the side facing the inside of the undergarment and the “outer side” of an element is the side facing the outside of the undergarment.

The brief portion 12 may also comprise an elastic waist band 26 and elastic borders (not shown) around the leg openings 22. The brief portion 12 could be made of materials in addition to the first material, for instance, it could be made of several different fabrics including the first material. However, according to the present invention, the brief portion 12 is made mostly of the first material (i.e. greater than 50% of the brief portion 12).

The crotch area 20 comprises a moisture control pad 30 which is disposed in a least the crotch area 20. The moisture control pad 30 extends continuously from the front side of the crotch area 20 to the underside of the crotch area 20 between the legs, to the seat rear 16 side of the crotch area 20. The pad 30 could extend upward beyond just the crotch area 20, but is preferably limited to the crotch area 20 as shown in FIGS. 1 and 2.

The moisture control pad 30 comprises a first layer 32 formed of a non-woven, absorbent material, and a second layer 34 which is moisture impermeable. The first layer 32 absorbs the moisture from the wearer's bodily fluids and preferably contains the moisture until the undergarment 10 is washed. The first layer 32 may be formed one or more thin layers of a non-woven, absorbent material which is reusable and launderable.

The second layer 34 is disposed on the outer side of the first layer 32 and covers substantially the entire outer side of the first layer 32. The second layer 34 is formed of a moisture and water vapor impermeable material. In a first embodiment of the undergarment 10 according to the present invention, the second layer 32 is made of a portion of the first material in the crotch area 20 which has been made moisture impermeable through a Teflon® treatment called the Repel Teflon treatment from DuPont Corporation. The Repel Teflon treatment is a nano-technology treatment in which a molecular barrier is formed at the nanoscale around individual fabric fibers. For repellent and impermeability performance, this barrier lowers the critical surface tension of the fabric so it repels most water-and oil-based liquids.

In another embodiment of the undergarment 10 according to the present invention, the second layer 34 may comprise a separate moisture impermeable protective layer such as a layer of impermeable polymer film and a layer of the first material on the outer side of the polymer film. The layer of the first material on the outer side of the polymer film may be an integral portion of the first material making up the brief portion 12, or it may be a separate piece of material formed of the first material. The polymer film may be attached to any part of the pad 30, but is preferably attached to the first material of the brief portion 12 in the crotch area 20. For example, the polymer film may be heat fused to the first material of the brief portion 12 in the crotch area 20. As described below, where the brief portion 12 does not have any material in the crotch area 20 where the pad 30 is placed, the second layer is made of separate fabric layer formed of the first material, and the protective layer is provided on the inner side of this fabric layer. The second layer 34 may then be attached to the remainder of the brief portion 12. brief portion as also described below. Where the first material of the brief portion 12 is an integral part of the material of the brief portion 12, the protective layer is attached, such as by heat fusing, bonding or other suitable method, to the inner side of the layer of first material of the brief portion 12 in the crotch area 20. The attachment of the protective layer to the first material of the brief portion may be done prior to assembling the undergarment 10 on a piece of the first material, or it can be done at any convenient point in the assembly process.

Finally, a third layer 36 formed of the first material is also provided in the crotch area 20 on the inner side of the first layer 32 which gives the crotch area 20 a look and feel that is substantially the same as the majority of the undergarment 10. The third layer 36 is disposed over any exposed portion(s) of the first layer 32. Thus, the only surfaces in contact with the wearer, including the inside and outside of the undergarment 10, are comprised of the first material.

There are various different configurations in which the first layer 32, second layer 34 and third layer 36 may be configured and assembled in the undergarment 10. The components of the undergarment 10 may be assembled to each other by stitching, adhesive, bonding, or other suitable means known to those of ordinary skill in the art. As several non-limiting examples of the various configurations, the third layer 36 may be an integral part of the material of the brief portion 12, and the first layer 32 and second layer 34 are then attached to the brief portion 12 in the crotch area 20. The first layer 32 and second layer 34 may be attached to each other prior to attaching them to the brief portion 12, or they may be attached separately one after the other.

Alternatively, the brief portion 12 may not have any material in the crotch area 20 where the pad 30 is placed, and the first layer 32, second layer 34 and third layer 36 may be attached to the remainder of the brief portion 12. Again, two or more of the first layer 32, second layer 34 and third layer 36 may be attached to each other prior to assembling them to the brief portion 12, or they be attached separately.

In another configuration of the undergarment 10, the second layer 34 may be an integral part of the first material making up the brief portion 12 which has been made moisture impermeable, such as by the Teflon treatment described above. Alternatively, the second layer 34 may be a separate piece of material formed of the first material made moisture impermeable. In such a configuration, the third layer 36 can be an integral part of the first material making up the brief portion 12, and first layer 32 and second layer 34 may be attached to the brief portion 12 on the outer side of the third layer 36. Or, the brief portion 12 can be made without material in the crotch area 20 where the pad 30 is located, in which case the third layer 36 is a separate piece of the first material. Then, the first layer 32, second layer 34, and third layer 36 can be assembled to the undergarment 10 in any of the applicable manners described above.

The desirable properties and specific materials for the first material, first layer 32 and second layer 34 will now be described in greater detail. The first material makes up the majority of the brief portion 12, the third layer 36 and is also the base material for the second layer 34. The first material is preferably a comfortable, soft, woven fabric which is amenable to being made moisture and vapor impermeable with the Repel Teflon treatment. It is also desirable that the first material be stretchable and elastic so that it forms well to the wearer's body size and shape. It is also desirable that the first material, as with all of the materials of the undergarment 10, have good antimicrobial and odor reducing properties. It is also desirable that the first material breathe and also relatively easily allow bodily liquids to pass through to the absorbent first layer 32. The first material should also retain these favorable characteristics, or minimize their degradation, after a reasonable number of launderings (for example, 5, 10 or 25 washings). Examples of several suitable materials for the first material, include without limitation, micro fabrics such as a super micro polyester and Spandex blend.

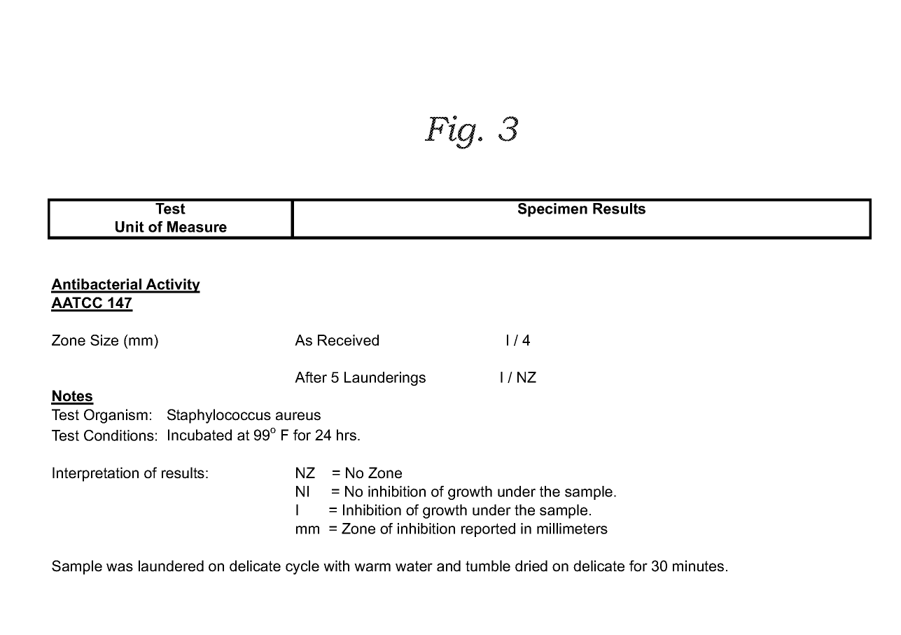

A suitable super micro polyester and Spandex blends includes a woven fabric composed of thread having about 94.4% super micro polyester and about 5.6% Spandex material (hereinafter referred to as the “super micro poly/Spandex material). Numerous tests were performed on samples of this fabric to determine its suitability as a first material in the undergarment 10. The results of this testing are shown in FIGS. 3-10, which will now be discussed in detail.

FIG. 3 shows the results of the antibacterial tests which were performed on the super micro poly/Spandex material according to the AATCC 147 standard test (American Association of Textile Chemists and Colorists). Under this test, the fabric is incubated at 99° F. for 24 hours with staphylococcus aureus bacteria. FIG. 2 indicates that the material, prior to any laundering, inhibits bacterial growth within a zone size of 4 mm from the fabric. After 5 launderings, the antibacterial properties of the fabric have diminished, but still there is inhibition of growth under the sample. In other words, the fabric remained antibacterial in that it did now allow bacteria to form on or under the fabric during the 24 hour incubation period.

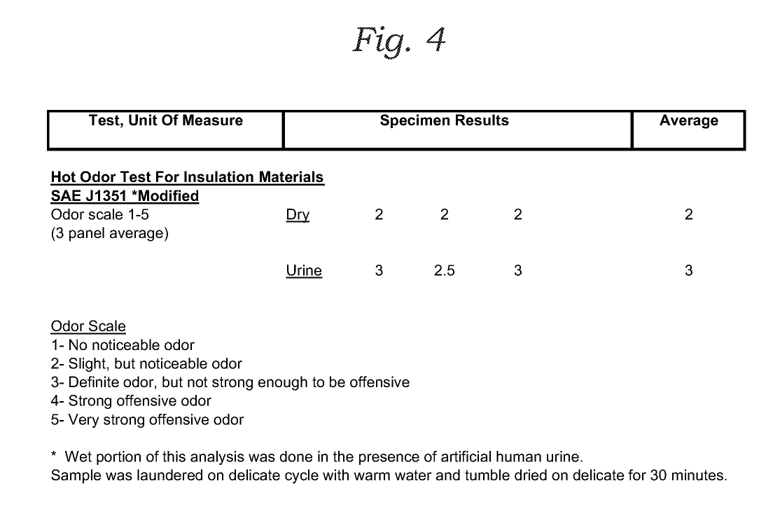

Turning to FIG. 4, the table shows the results of the Hot Odor Test on the super micro poly/Spandex material. The Hot Odor Test was performed according to SAE J1351 *Modified standard test (the modification consisted of using artificial human urine for the Wet portion of the test) (Society of Automotive Engineers). Three specimens were tested and the results were averaged. The results indicate that the super micro poly/Spandex material has a slight odor (average 2 rating) when dry. The urine test (wet test) had an average of 3 indicating a definite odor, but not strong enough to be offensive. Thus, it can be seen that the super micro poly/Spandex material has at least moderate odor reducing characteristics.

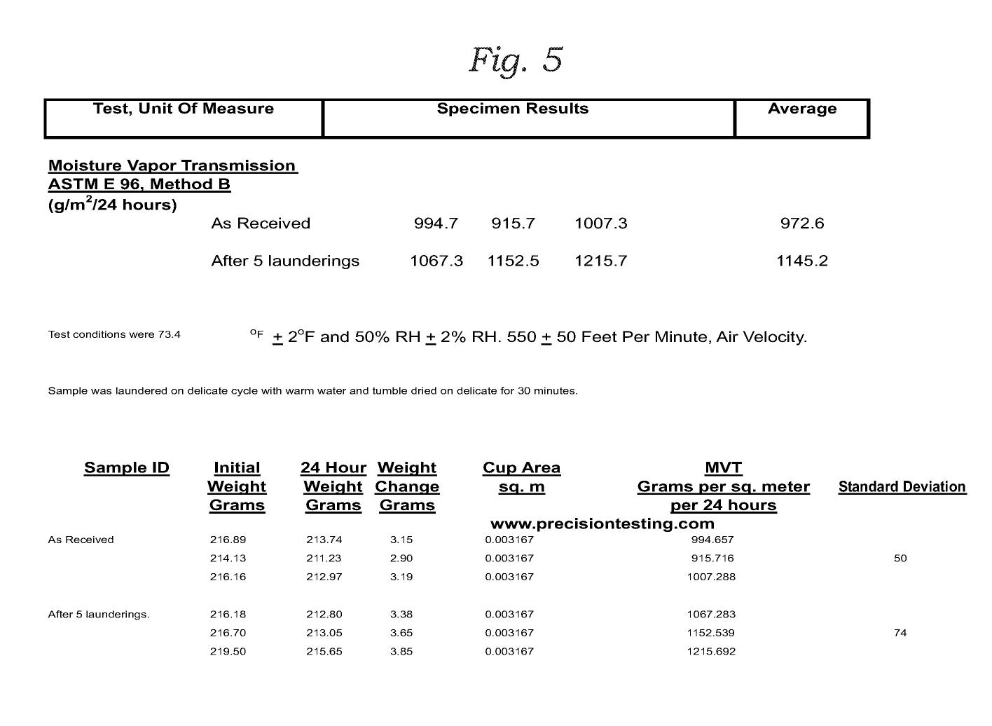

The Moisture Vapor Transmission test was performed on the super micro poly/Spandex material pursuant to ASTM E 96, Method B, the results of which are shown in FIG. 5. This test measures the porous nature of fabric, or how well it breathes, thus allowing air flow between the fibers. The unlaundered samples had an average rating of 972.6. The average rating for the samples after 5 launderings was 1145.2, or an improvement of about 15%. Thus, the breathability of the super micro poly/Spandex actually improves with laundering.

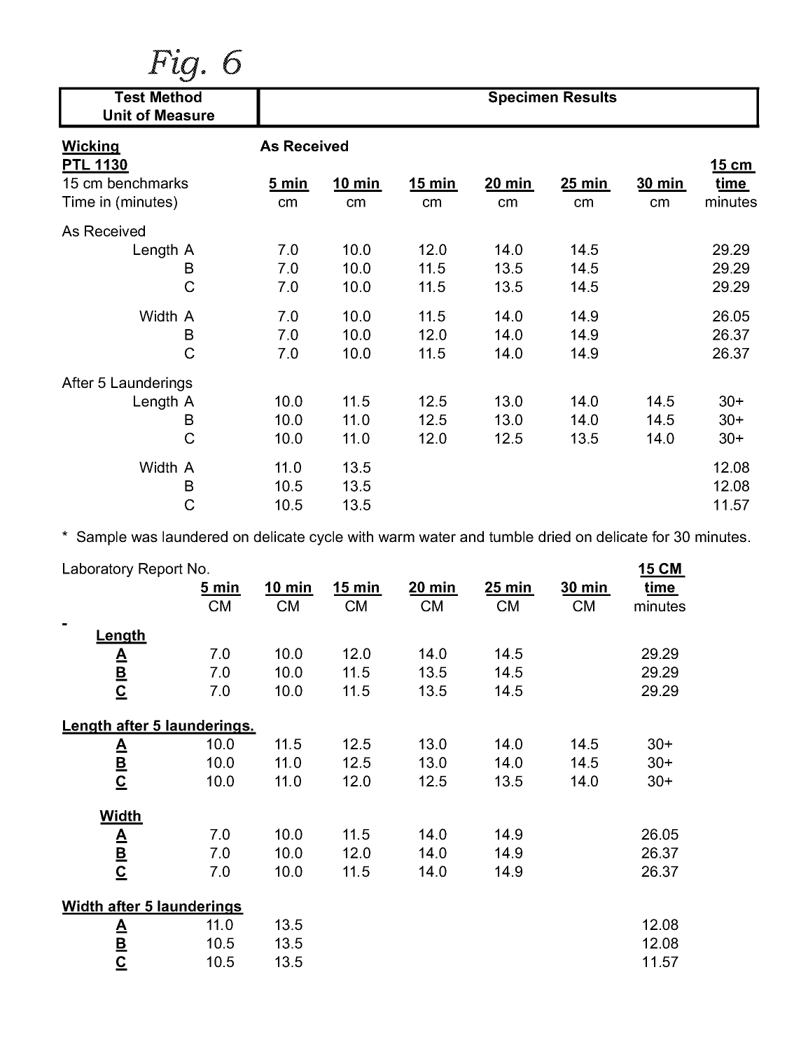

FIG. 6 shows the results of the PTL Wicking 1130 test on the super micro poly/Spandex material. This test examines the rate at which liquid wicks (i.e. moves) across the length and width of a sample of fabric. The results show that the liquid adequately wicks across the super micro poly/Spandex material, and that laundering does not significantly degrade the wicking properties of the material.

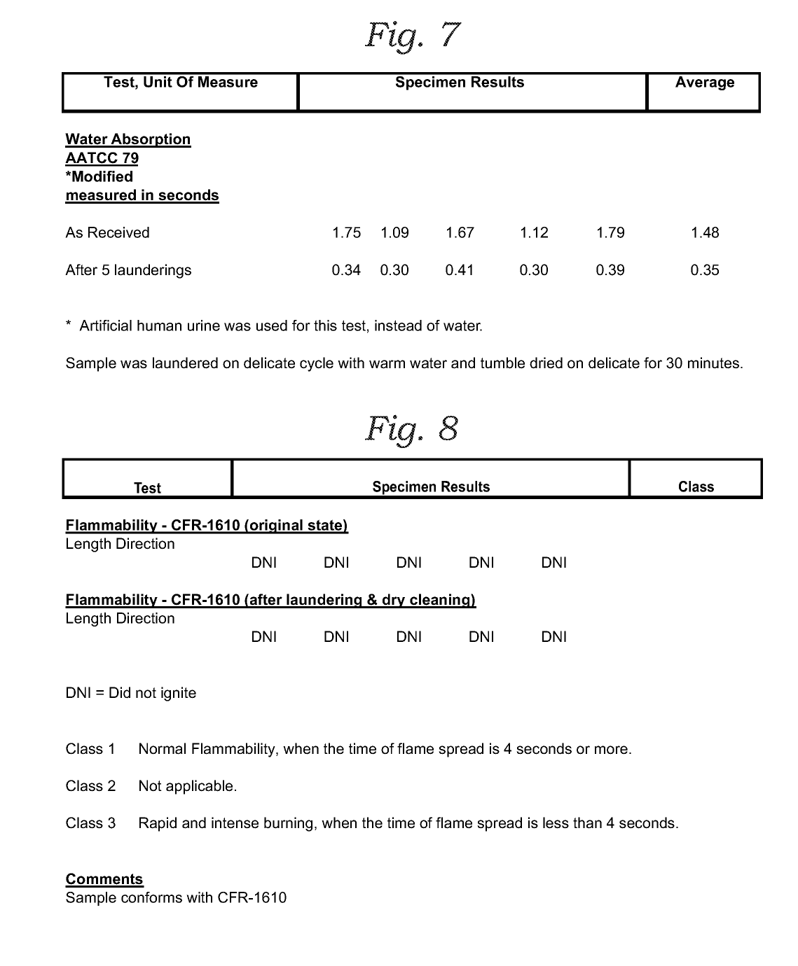

The results of the Water Absorption test are shown in FIG. 7. The Water Absorption test was performed pursuant to AATCC 79 *Modified (the modification consisted of using artificial human urine for the test, instead of water). This test measures the ability of the fabric to quickly dissipate liquid applied to the fabric. For the unlaundered material, the average time it took the fabric to dissipate the urine was 1.48 seconds. After the samples were laundered 5 times, the average time was reduced to 0.35 seconds. Thus, the super micro poly/Spandex exhibits good liquid dissipation characteristics which actually improve with laundering.

FIG. 8 shows the results of the Flammability Test for the super micro poly/Spandex material, which was performed according to CFR-1610. The results show that the super micro poly/Spandex material did not ignite and therefore conforms with CFR-1610. In other words, the fabric has excellent non-flammability properties.

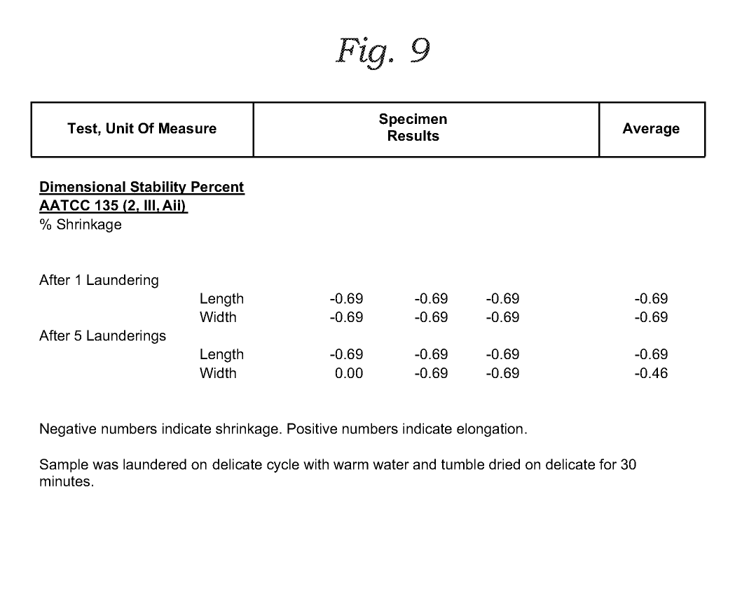

FIG. 9 shows the results of the Dimensional Stability Test pursuant to AATCC 135 (2, III, Aii) for the super micro poly/Spandex material. This test measures the tendency of the fabric to dimensionally change in the width and length due to laundering. The results in FIG. 9 show that the super micro poly/Spandex material shrinks very slightly (less than 1%) due to laundering, and that the material stabilizes and does not continue to shrink from multiple launderings.

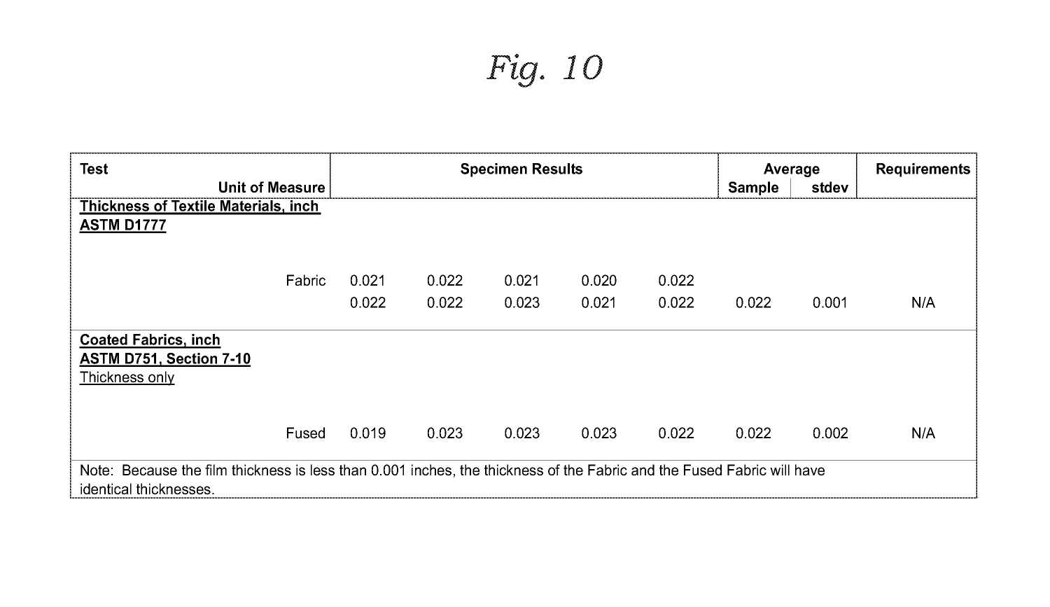

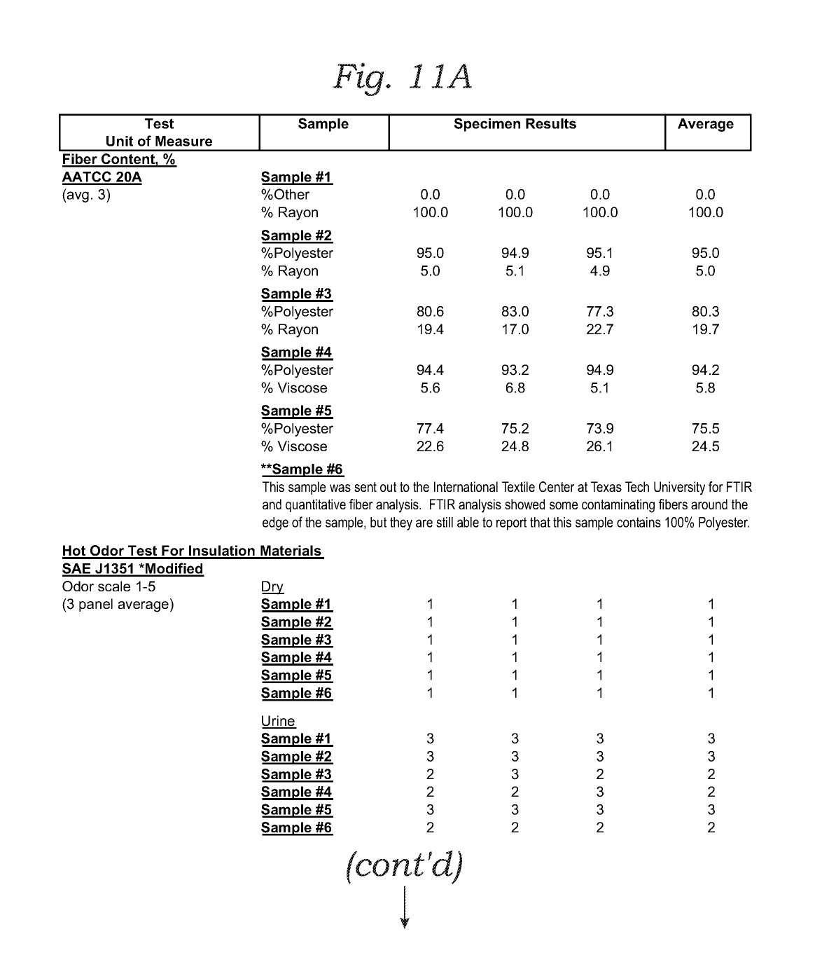

Finally, FIG. 10 shows the thickness of the super micro poly/Spandex material as measured pursuant to ATM D1777 (American Society for Testing Materials). The samples of fabric tested varied in thickness from 0.020 inch to 0.023 inch, and had an average thickness of 0.022 inch. FIG. 10 also shows the thickness of samples of the super micro poly/Spandex material having a polyurethane film heat fused to the material to make it moisture impermeable. The results show that the heat fused film does not add any appreciable thickness to the material.

The first layer 32 is a non-woven, absorbent material. It is primarily important that the material making up the first layer have the capacity to absorb an appreciable amount of bodily fluids. It is also desirable that the material have good antimicrobial and low odor emission characteristics. The first layer 32 should also retain these favorable characteristics, or minimize their degradation, after a reasonable number of launderings (for example, 5, 10 or 25 washings). Examples of several suitable materials for the first material, include without limitation, a polyester and viscose fabric such as those available from Texel, or a polyester and rayon fabric such as those available from National Nonwovens.

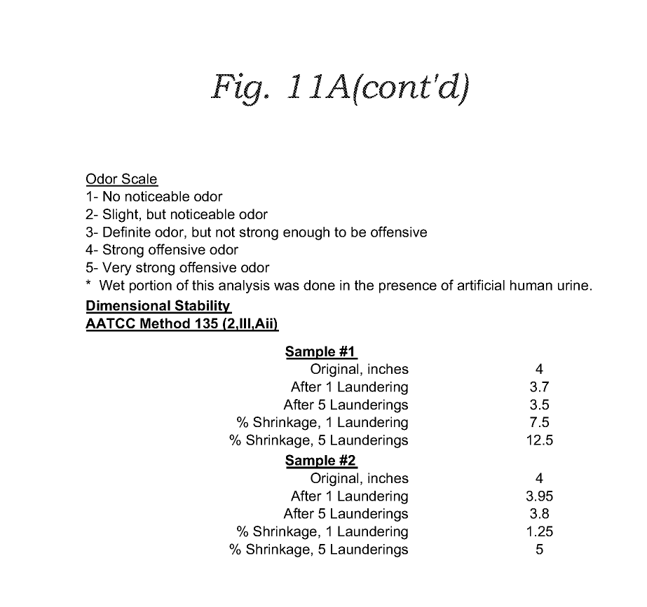

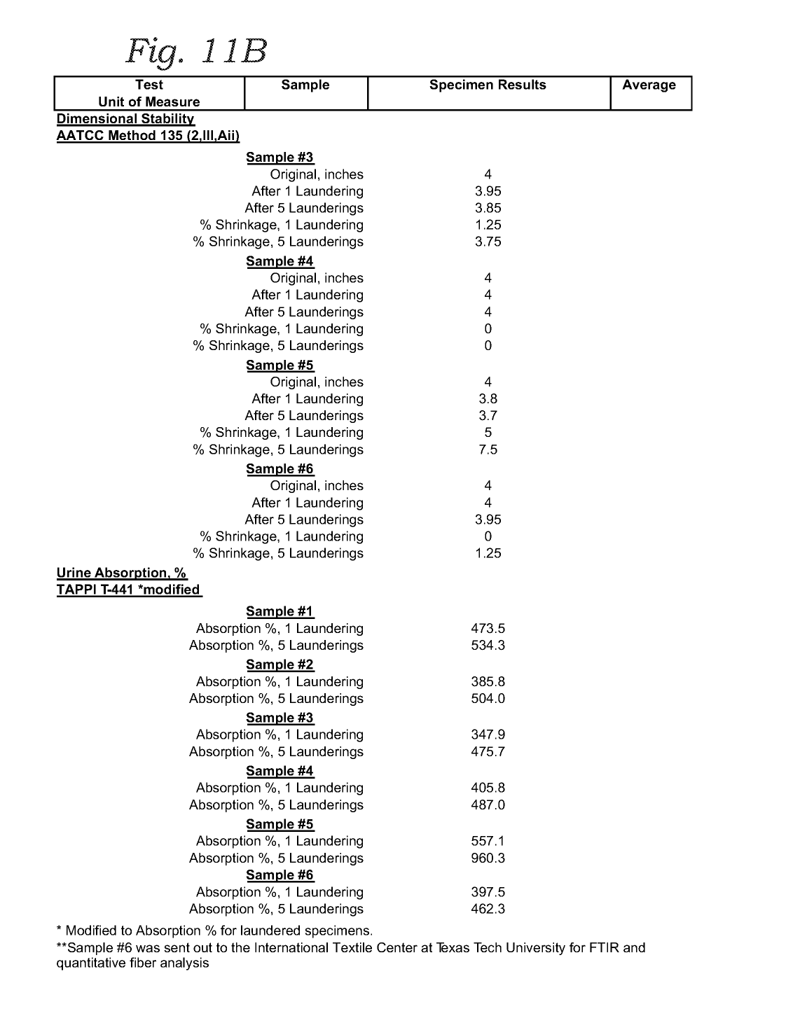

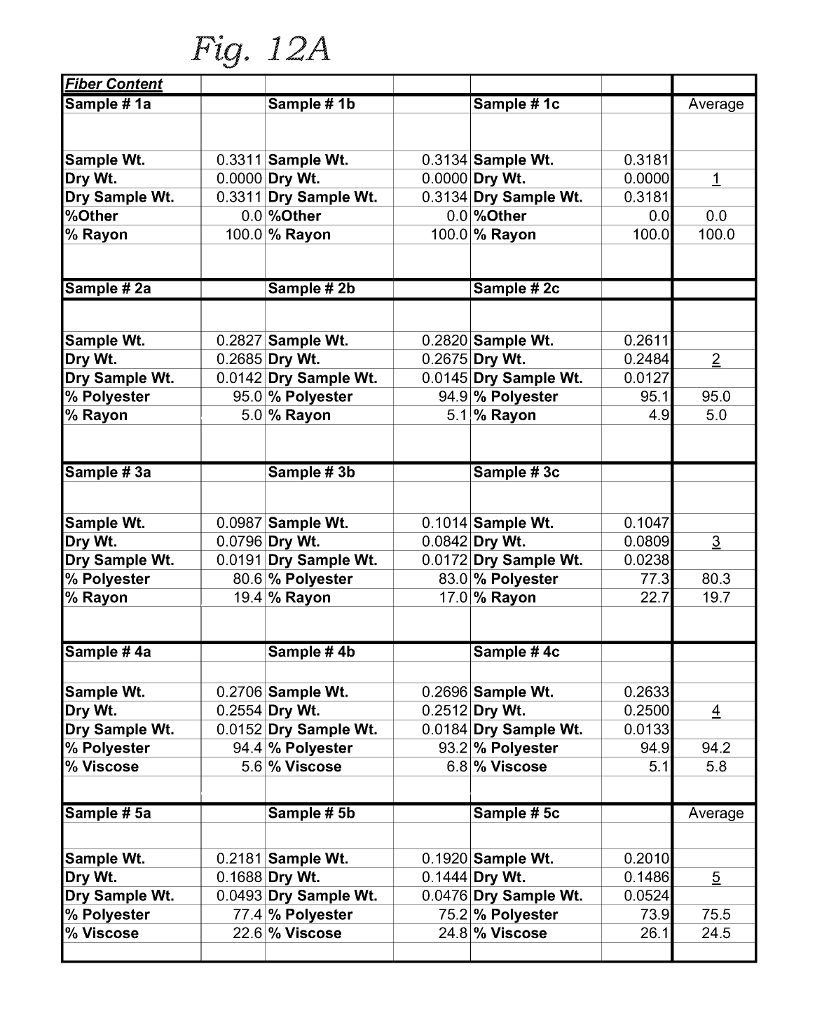

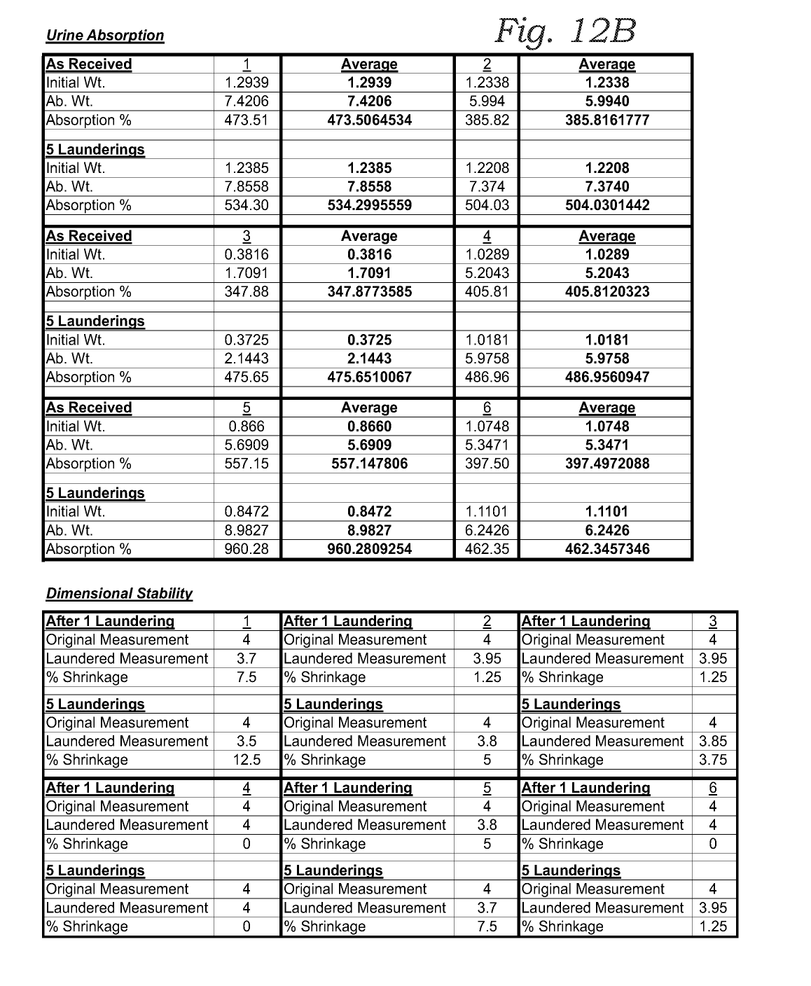

Several suitable non-woven fabrics for forming the first layer 32 were chosen and tested. The results of these tests are shown in FIGS. 11-13. For the sake of brevity, only the results for the fabric comprised of about 95% polyester and about 5% viscose (hereinafter referred to as the “poly/viscose material”) will be discussed herein, which is identified as Fabric #4 in FIGS. 11-12. The comparative characteristics of each of the other tested fabrics can be seen by reference to FIGS. 11-12.

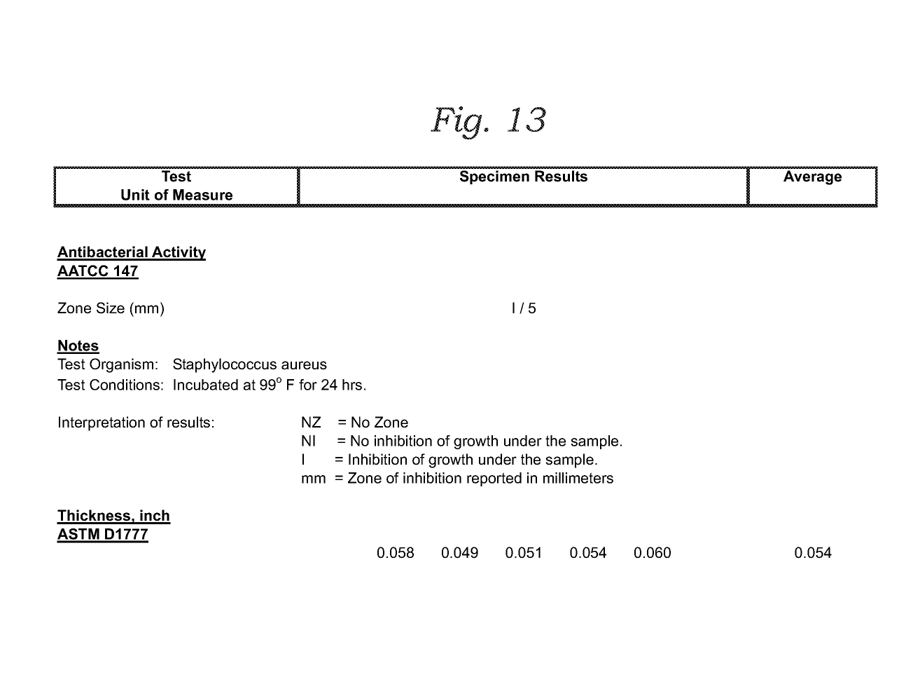

As shown in FIG. 13, the poly/viscose material has good antibacterial activity with an inhibition zone of 5 mm. The Hot Odor test results shown in FIG. 11A indicate that the average dry rating was 1 (no noticeable odor) and the average wet rating was 2 (slight, but noticeable odor). Thus, the poly/viscose has acceptable odor retention properties.

The urine absorption test was run on the poly/viscose material according to TAPPI T-441 *Modified (only the results reported were modified to % absorption and milliliters (ml) absorbed) (Technical Association of Pulp and Paper Industry). Prior to laundering, the poly/viscose sample absorbed 4.2 ml and improved to 5.0 ml after 5 launderings. This shows good absorption capacity and the ability to withstand laundering.

The test results for the poly/viscose material also show that it has excellent dimensional stability. Lastly, FIG. 13 shows the thickness of the a single layer of the tested poly/viscose material varied from 0.049 inch to 0.060 inch, with an average thickness of 0.054 inch. It can be reasonably assumed that two layers of the poly/viscose material will have twice this thickness, and so on for each additional layer.

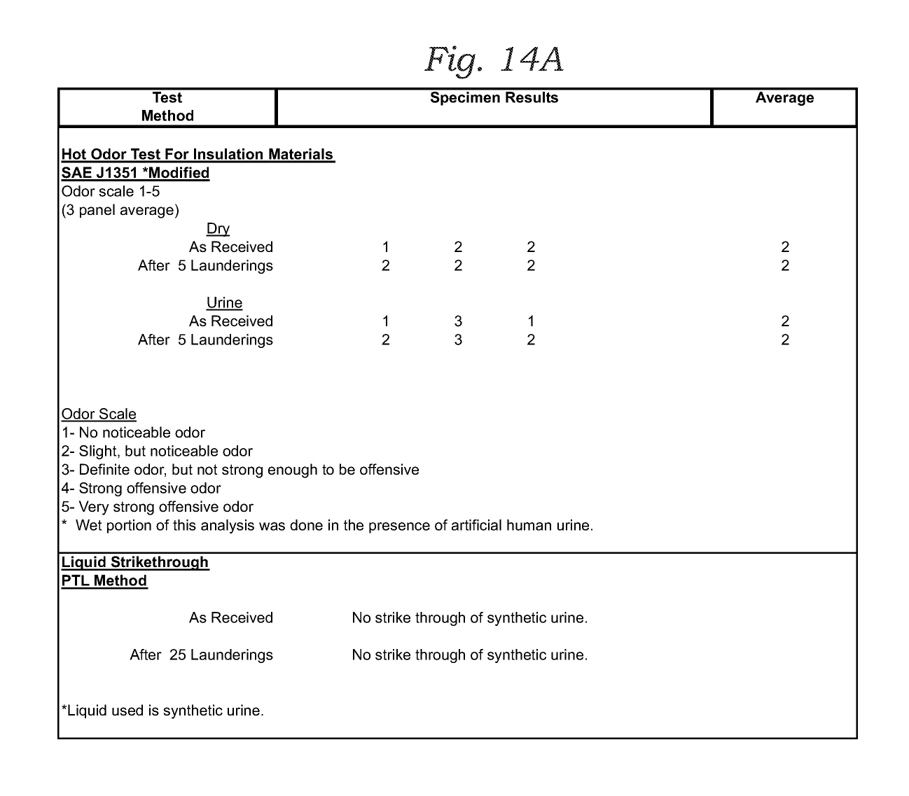

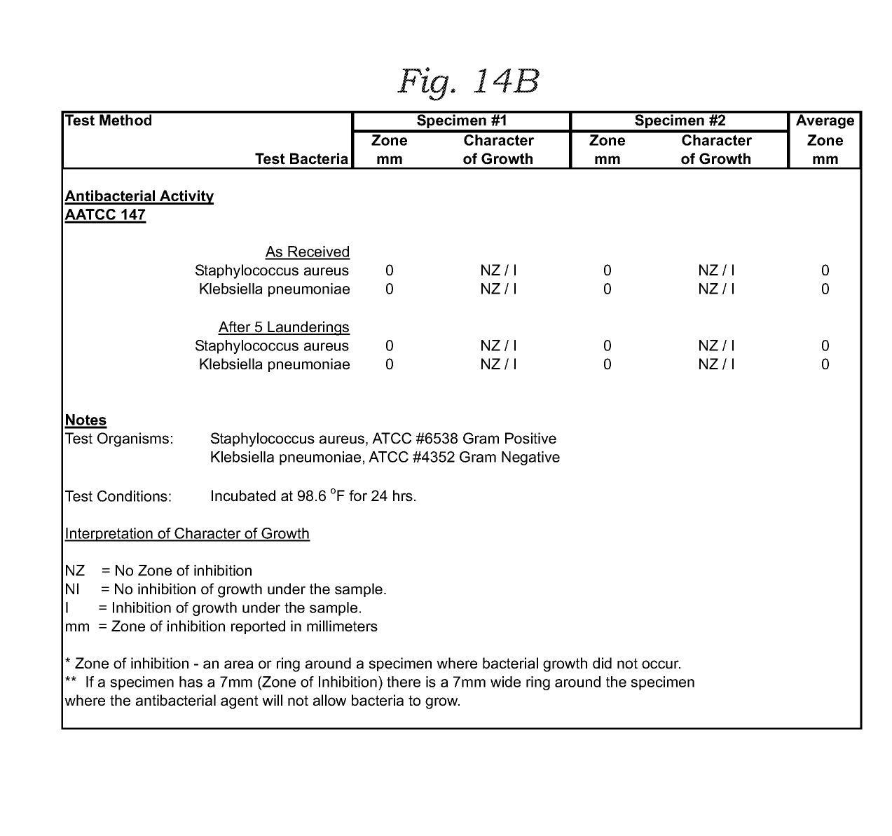

In the first embodiment of the undergarment 10 according to the present invention described above, the second layer 32 is made of a portion of the first material which has been made moisture impermeable through a Teflon® treatment called the Repel Teflon treatment from DuPont Corporation. Thus, FIGS. 14A-14B show test results for a material comprising the super micro poly/Spandex material which has been treated with the Repel Teflon treatment (hereinafter referred to as the “Teflon poly/Spandex” material). FIGS. 14A-14B indicate that the Teflon poly/Spandex material has good odor retention characteristics and good antibacterial properties. And very importantly, the Teflon poly/Spandex material prevents liquid strikethrough (i.e. the material is moisture impermeable) as tested pursuant to the PTL 1150 standard test. Moreover, the Teflon poly/Spandex material remained absolutely leak-proof even after 25 launderings. Finally, the Repel Teflon treatment adds no thickness to the super micro poly/Spandex material such that the thickness of the Teflon poly/Spandex material is the same as the super micro poly/Spandex material.

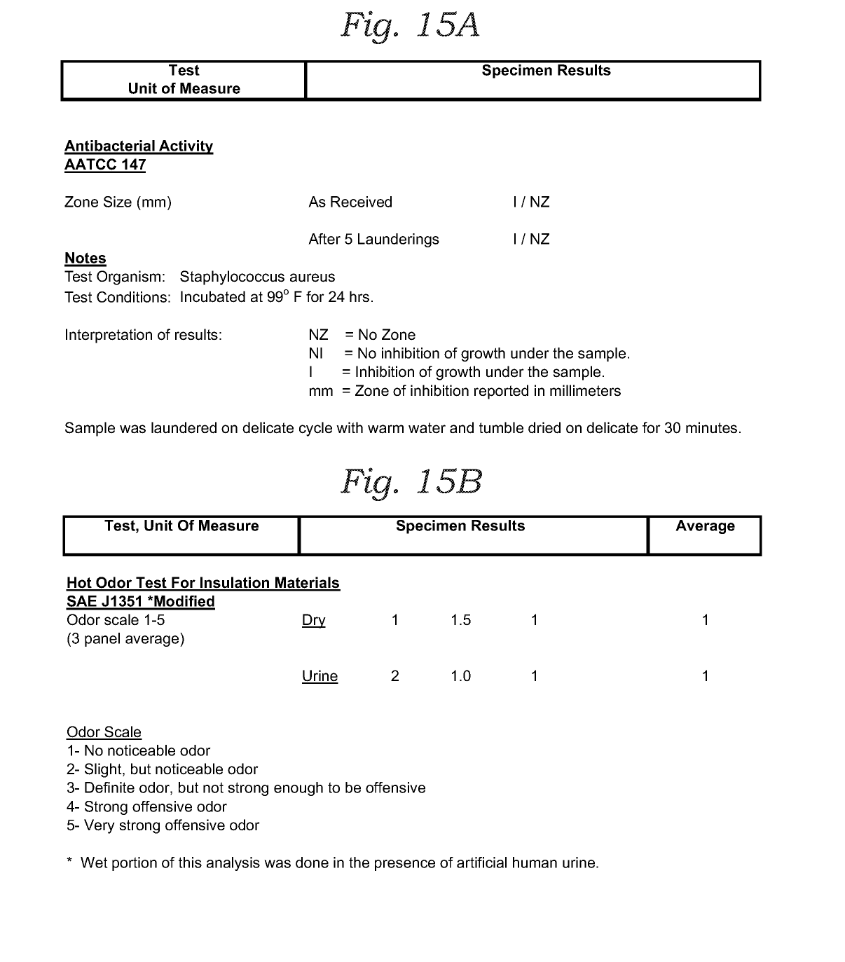

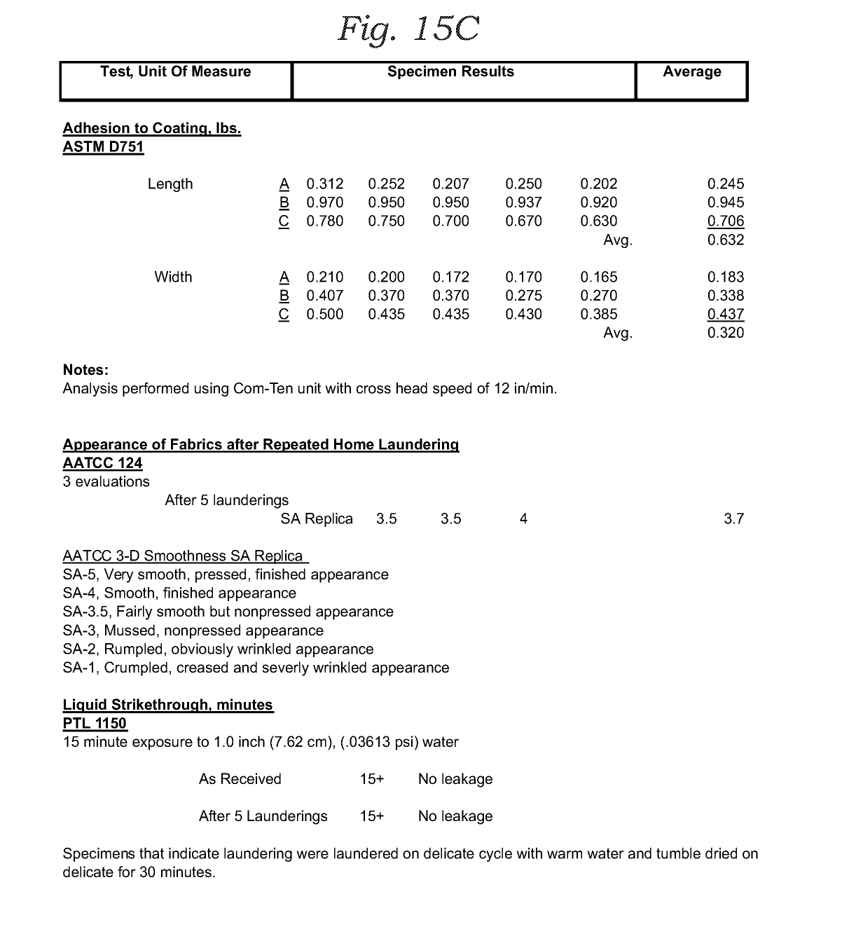

In one alterative embodiment of the undergarment 10 according to the present invention, the second layer 34 may be made of a portion of the first material with a polymer film heat fused to the inner side of the portion of the first material. According to this embodiment, FIGS. 15A-15C show test results for the super micro poly/Spandex material which has a 100% polyurethane film heat fused to its inner side. The test results in FIGS. 15A-15C show that this material has suitable antibacterial and odor properties and is also impermeable to moisture, even after being laundered.

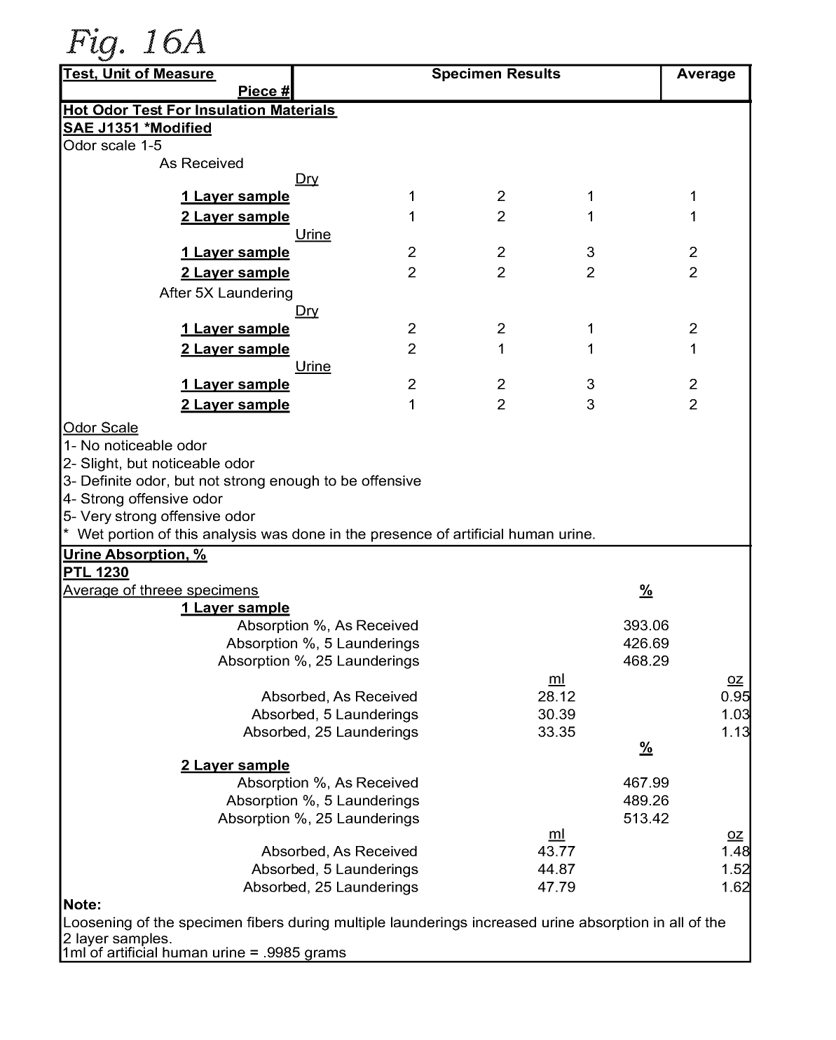

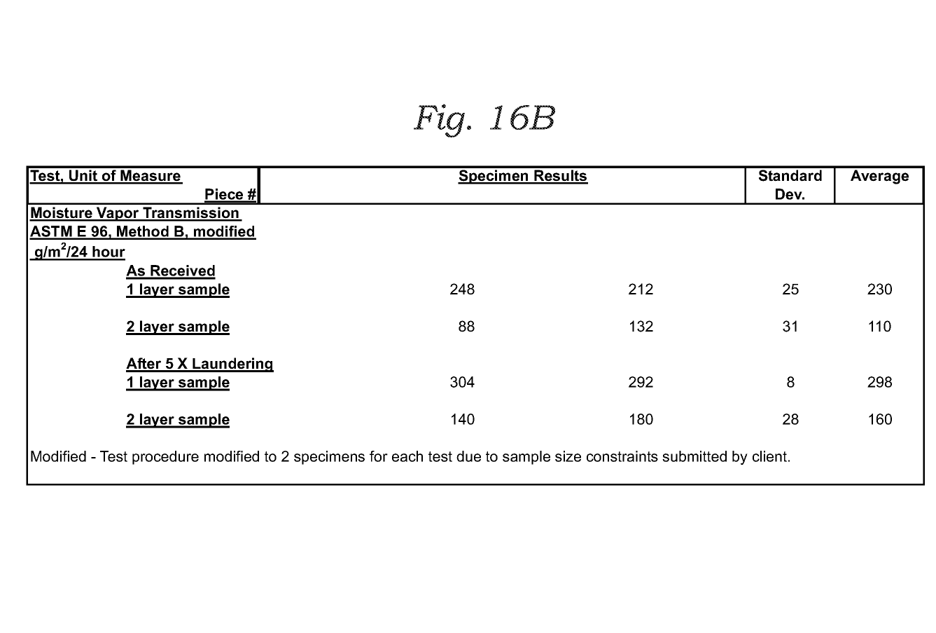

Testing was also conducted on a complete undergarment 10 according to the present invention with the first material comprised of the super micro poly/Spandex material, the first layer 32 comprised of one and two layers, respectively, of the poly/viscose material, the second layer comprised of the Teflon poly/Spandex material, and the third layer comprised of the super micro poly/Spandex material. The results of this testing are included in FIGS. 16A-16B. The results show that the undergarment 10 has good odor, absorption and moisture vapor transmission properties for both one layer and two layers of poly/viscose material. Moreover, the thickness of the moisture control pad 30 for the undergarment 10 with one layer of poly/viscose material is only about 0.08 inch (and the total thickness of the crotch area 20 which includes the third layer 36 is only about 0.010 inch). And for two layers of poly/viscose material, the thickness of the pad 30 is only about 0.16 inch (and the total thickness of the crotch area 20 which includes the third layer 36 is only about 0.18 inch).

While the present invention has been fully described above with particularity and detail in connection with what is presently deemed to be the invention, it will be apparent to those of ordinary skill in the art that many modifications thereof may be made without departing from the principles and concepts set forth herein. Hence, the proper scope of the present invention should be determined only by the broadest interpretation of the appended claims so as to encompass all such modifications and equivalents.

What is claimed is:

1. A reusable, launderable, moisture protective undergarment, comprising:
a brief portion made mostly of a first material, the brief portion having an inside surface which is in contact with a wearer of said undergarment and an outside surface which faces outward from the wearer, the brief portion having a waist opening, two leg openings and a crotch area, said crotch area comprising;
a moisture protection pad disposed in at least said crotch area, said pad comprising a first layer and a second layer covering substantially the entire outer side of said first layer, said first layer disposed on the inner side of said second layer, said first layer formed of a non-woven, absorbent material, and said second layer formed of said first material which has been made moisture impermeable through a Teflon® treatment, said pad having a total thickness of less than 0.10 inches; and
a third layer formed of said first material covering the entire inner side of said first layer.

2. The undergarment of claim 1, wherein said Teflon® treatment comprises the Repel Teflon treatment from DuPont.

3. The undergarment of claim 1, wherein said first material is formed of super micro-polyester and Spandex material.

4. The undergarment of claim 1, wherein said first layer is formed of two layers of a non-woven, absorbent material.

5. The undergarment of claim 1, wherein said first layer is formed of a single layer of a non-woven, absorbent material.

6. The undergarment of claim 1, wherein said first layer is disposed on the outside surface of said brief portion.

7. The undergarment of claim 1, wherein said first layer is disposed on the inside surface of said brief portion.

8. The undergarment of claim 1, wherein said first layer is formed of two layers of a blended polyester and viscose material, and said first material is formed of a super micro-polyester material.

9. The undergarment of claim 1, wherein said third layer is an integral part of the first material comprising the brief portion.

10. A reusable, launderable, moisture protective undergarment, comprising:
a brief portion at least partially made of a first material, the brief portion having an inside surface which is in contact with a wearer of said undergarment and an outside surface which faces outward from the wearer, the brief portion having a waist opening, two leg openings and a crotch area, said crotch area comprising:
a moisture protection pad disposed in at least said crotch area, said pad comprising a first layer formed of a non-woven, absorbent material, and a second layer formed of a moisture impermeable material; said pad having a total thickness of less than 0.13 inches; and
a third layer formed of said first material covering the entire inner side of said first layer.

11. The undergarment of claim 10, wherein said second layer comprises a fabric layer made of fabric and a protective layer formed of a moisture impermeable material.

12. The undergarment of claim 11, wherein said inner protective layer is heat fused to said fabric layer.

13. The undergarment of claim 10, wherein said first layer is formed of a blended polyester and viscose material.

14. The undergarment of claim 10, wherein said pad is disposed on the outside surface of said brief portion.

15. The undergarment of claim 10, wherein said pad is disposed on the inside surface of said brief portion.

16. The undergarment of claim 10, wherein said pad comprises the crotch portion of said brief portion.

17. The undergarment of claim 12, wherein said first layer is formed of a blended polyester and viscose material, said second layer comprises a inner protective layer and an outer fabric layer wherein said inner protective layer is a polyurethane film heat fused to said outer fabric layer, and said first material and said outer fabric layer are formed of a super micro-polyester and spandex material.

18. The undergarment of claim 12, wherein said third layer is an integral part of the first material comprising the brief portion.
